(19) United States
(12) Patent Application Publication
(10) Pub. No.: US 2024/0139544 A1
(43) Pub. Date: May 2, 2024

(54) System and Method for Radiopharmaceutical Treatment Outcome Prediction Using Machine Learning

(71) Applicant: BAMF Health, Inc., New York, NY (US)

(72) Inventors: Eric Walden Brunner, Dallas, TX (US); Jeffrey Lee VanOss, Kentwood, MI (US); Stephen Moore, Singapore (SG); Gowtham Murugesan, Houston, TX (US); Anthony Chang, Grand Rapids, MI (US)

(73) Assignee: BAMF Health, Inc., (omitted)

(21) Appl. No.: 18/119,723

(22) Filed: Mar. 9, 2023

Related U.S. Application Data
(63) Continuation-in-part of application No. 17/190,301, filed on Mar. 2, 2021.
(60) Provisional application No. 63/423,999, filed on Nov. 9, 2022.
(60) Provisional application No. 63/110,491, filed on Nov. 6, 2020.

(51) Int. Cl. A61N 5/10; G16H 10/60; G16H 20/40; G16H 50/30

(52) U.S. Cl. CPC A61N 5/1039; A61N 5/1031; A61N 5/1038; G16H 10/60; G16H 50/30; A61N 2005/1041; G16H 20/40

(57) ABSTRACT

Methods and systems are described and claimed that employ machine learning data processing models to generate for a cancer patient being treated with a radiopharmaceutical a predicted Absorbed Dose Map (ADM), a predicted PET scan or a predicted outcome parameter for an administered dose of the radiopharmaceutical.

### PRIORITY CLAIM

This is a utility patent application that claims priority to U.S. Provisional Application No. 63/423,999 filed on Nov. 9, 2022, which is incorporated herein by reference and as a continuation in part to U.S. patent Ser. No. 17/190,301 filed on Mar. 2, 2021 which is incorporated herein by reference, which further claims priority to Provisional Application 63/110,491 filed on Nov. 6, 2020 which is also incorporated by reference.

### FIELD OF INVENTION

This relates to image processing and machine learning in a specific way to enable and improve radiopharmaceutical therapy analysis and treatment planning.

### BACKGROUND

Radiopharmaceutical therapy involves the targeted delivery of radiation to tumour cells. A Radiopharmaceutical is a drug that can be used either for diagnostic or therapeutic purposes. In one embodiment, it is composed of a radioisotope of an element bonded within an organic molecule. Other embodiments may be non-organic molecules. The bound molecule conveys the radioisotope to specific organs, tissues or cells. The radioisotope is selected for its properties and its diagnostic utility or treatment utility. This treatment approach is distinguished from external beam radiotherapy and brachytherapy in that the radiation is delivered by unencapsulated radionuclides through an injectable solution or suspension that is distributed throughout the body, rather than localized to the site of injection. Radiopharmaceutical dosimetry describes the interaction between the energy deposition associated with a radiopharmaceutical's emissions and the patient's body and helps to guide optimal clinical use of radiopharmaceuticals. More specifically current dosimetry reports used in today's medical field are to quantify administered amounts of radioactivity to the absorbed radiation dose in tumours, organs, or the whole body. Dosimetry is important for dose correlation with clinical results, and in some instances, for treatment planning to avoid excess toxicity. Currently the generation of a dosimetry report is a manual effort by a radiologist which is a time consuming process. Current conventional imaging (CT, MM, bone scan) are mostly unable to detect disease recurrence (when tumours are small) for example in Prostate cancer if PSA levels are low. Prostate PET ligands fill this diagnostic void of occult disease and allow a chance at early localisation and detection.

Accurate dosimetry is needed in systemic internal radiation treatments (SIRT) to ensure that absorbed dose (AD) limits to organs at risk are not exceeded over the lifetime of the patient and to ensure that adequate radiation doses are absorbed by all regions within a tumor to facilitate a sufficient disease response outcome. Dosimetry workflows vary from clinic to clinic and are not standardized within the nuclear medicine field. All dosimetry workflows require the acquisition of one diagnostic CT scan from which organ and tumor masses are derived and used in the calculation of AD. A series of planar scintigraphy scans and diagnostic CT scan (101) may be combined to create a planar dosimetry workflow. Similarly, the planar scans and diagnostic CT scan may be utilized in combination with a SPECT scan (102) performed at the same time as one planar scan to create a hybrid dosimetry workflow (104). Furthermore, the diagnostic CT scan and a series of SPECT scans (103) at various times after treatment may be combined to create a SPECT dosimetry workflow. (105) All dosimetry workflows require the acquisition of multiple post-injection scans (PIS), which are typically SPECT or planar scans that detect the emission of the therapeutic radiopharmaceutical that has been administered to track temporal changes in the radiopharmaceutical's emission intensities within the body following injection. PIS can be two-dimensional in the case of planar scans or three-dimensional in the case of SPECT scans. Multiple planar scans are required for planar dosimetry, multiple planar scans and one SPECT scan are required for hybrid dosimetry, and multiple SPECT scans are required for SPECT dosimetry.

In pixel-based (2D) or voxel-based (3D) dosimetry workflows, PIS are aligned to one another using registration algorithms such that pixels or voxels within the same regions of the body overlay across scans obtained at different post-injection times. Dose-rate maps, which are images, are then generated from the registered planar or SPECT scan images through the local-deposition method (201), dose-point kernels (202), or Monte Carlo simulations (203) by determining the rates at which radiation is deposited in neighboring pixels or voxels based on the activity occurring in a source pixel or voxel. The does rate map indicates the rate at which radiation is decaying at a point in the image or volume at a point in time. The dose rate maps (301) are aligned so that a location (x,y) in the nth 2D image corresponds to the same location in n+1th image. And likewise for locations (x,y,z) of a voxel in a 3D image. The images may be stored in a computer system as a data structure comprised of data representing a pixel or voxel location in the image and a corresponding one or more color and corresponding intensity values for that pixel or voxel. The series of n images (301) represent dose rate maps at a sequence of times after the dosage has been applied. For each point, whether a pixel in 2D or a voxel in 3D, a dose rate curve plotting dosage rate versus time may be determined. (302) The curve may be stored in a computer system by storing a data structure comprised of nodes, where for each node, the function value and corresponding to each discrete input variable may be stored as elements. That curve can then be integrated using numerical approximations of calculus applied to the function values to calculate a total dosage delivered to that point. (303) The integrated values for all of the points may be presented as an image showing an absorbed dose map (304).

The absorbed dose is how much radiation was actually absorbed by the tissue at a location. For a given point location x,y or x,y,z, the absorbed dose for that point is the integration of the dose rates at that local point over the time that the radio pharmaceutical is present in the patient body. The dose rate map can be converted into an absorbed dose map by several techniques. Dose-rate curves (DRC) for every pixel or voxel are then constructed by plotting the dose-rate within every pixel or voxel against the duration of time occurring in between injection and the time of PIS acquisition. DRC are then fitted with a mathematical equation or combination of equations wherein the area under the fitted mathematical equation(s) represents the absorbed dose for a single pixel or voxel for an entire cycle of treatment. The shape of the DRC may be different for different tissues in a patient because the absorption amount is dependent on which type of tissue is being treated. Different tissues exhibit different radiation absorption characteristics. The absorbed dose values are then mapped backed into an aligned matrix to produce an absorbed dose map (ADM) for an entire cycle of treatment. Determining the absorbed dose to any organ or tumor proceeds by annotating a segmentation mask data object on top of the ADM and summing the AD values for every pixel or voxel within the annotation. The Absorbed Dose Map image (401) can be segmented by using an image or 3D scan that has located the organs of the patient. (402). The image data of the scan can be processed to detect the location of edges or regions in the image corresponding to an organ or tumor and that location data can then be used to determine the annotation of the segmentation mask. The computer can then sum the absorbed dose values for every pixel or voxel for each organ or tumor to calculate the absorbed dosage for that organ or tumor. (403)

SIRT are commonly administered in multiple cycles with several weeks spaced in between cycles (501, 502, 503). The determined AD value for any given organ for one cycle of treatment is summed with any previous or subsequent cycle's AD value to obtain the cumulative AD for that organ over a patient's lifetime. The cumulated AD is checked against recommended dose limits to avoid increasing the likelihood for onset of any adverse events related to that organ that arise from elevated cumulative AD. AD values and cumulative AD values for tumors, areas (2D) within those tumors, or volumes (3D) within those tumors is likely to be crucial to understanding tumor response to systemic internal radiation treatments.

### SUMMARY

Regardless of chosen dosimetry workflow, multiple PIS are required for accurate determination of AD values; however, the acquisition of multiple PIS can be burdensome on both the patient and clinical operating logistics. Several approximation methods have been proposed to reduce the required number of PIS but are not entirely valid for every patient or in every circumstance. Therefore, there is a need for an improved dosimetry process to reduce the number of PIS needed. In one embodiment of the invention, the system and method uses the pre-planned administered dose(s) values used in each cycle of treatment, patient health data set (PHD) and the diagnostic PET/CT scan data acquired before radio-therapeutic treatment, whether by injections alone or in combination with a reduced number of PIS as inputs into a machine learning model that is trained to predict the absorbed dosage at a location, in a segmented part of an image representing an organ or as a predicted Absorption Dose Map. The PHD may include clinical lab data like blood tests, treatment history, health history, demographic characteristics and genomic profiles, proteomic profiles, mRNA profiles, metabolomics, metagenomics, phenomics and transcriptomics.

This approach minimizes the total number of PIS required following each cycle of treatment for accurate AD determination. The diagnostic PET/CT scans in combination with or without other acquired PIS hereafter are referred to as input scans (IS). Scans are typically represented as an image file or a data structure comprised of elements representing pixel or voxel values along with tags, location data or other data relevant to the data element. In some embodiments, the scans may be dynamic scanning. For example, the PET scan image data may be a series of images representing a video depicting the diffusion of the PET marker into the patient when the PET scan technique was commenced. The training set for training the machine learning engine is a large set of known patient radiation treatment histories represented by data that represent a huge number of variables that create a large range of possible outcomes across the range of patients. The machine learning engine that is trained using these data inputs is able to use that base knowledge data to predict ADM data output, overall efficacy of the treatment or adverse effects as represented by numerical output parameters.

In an embodiment for generating predicted post injection PET scans, training a machine learning engine to make such a prediction requires using a set of known diagnostic PET or CT scans, corresponding known Patient Health Data and corresponding known post-treatment PET scans or data representing known treatment outcomes. One advantage of using actual PET scans to train the machine learning engine is that the post-treatment PET scan is done much later after the therapeutical radiopharmaceutical is administered, for example months later, whereas SPECT scans are typically made shortly the after the therapeutical radiopharmaceutical is administered, for example, hours or days, which it typically before any beneficial effect on the tumor can be detected. As a result, the SPECT scan does not show the treatment's effect on the tumor itself but a PET scan more likely does. The output of the trained machine learning engine may be a predicted PET scan displaying a predicted effect on the patient's tumor. The use of predicted PET scan output using the machine learning engine means that fewer SPECT scans are necessary. In addition, there is a need for a treatment planning system that uses the predictive capabilities of the trained machine learning engine to ascertain the expected results of a proposed radiation prescription and the likelihood of any adverse side effects of the treatment plan.

### DESCRIPTION OF THE FIGURES

The headings provided herein are for convenience only and do not necessarily affect the scope or meaning of the claimed invention. In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced (e.g., element 101 is first introduced and discussed with respect to FIG. 1).

FIG. 1a Flowchart depicting one embodiment of the claimed invention using Planar Dosimetry Workflow.

FIG. 1b. Flowchart depicting one embodiment of the claimed invention using Hybrid Dosimetry Workflow,

FIG. 1b. Flowchart depicting one embodiment of the claimed invention using SPECT Dosimetry Workflow.

FIG. 2a Flowchart depicting a second embodiment of the claimed invention using local deposition assumption.

FIG. 2b Flowchart depicting a second embodiment of the claimed invention using dose point kernel.

FIG. 2c Flowchart depicting a second embodiment of the claimed invention using Monte Carlo simulation.

FIG. 3 depicts a process for generating absorbed dose maps from aligned dose-rate maps

FIG. 4 depicts a process for determining absorbed dose values within organs from an absorbed dose map and segmentation masks

FIG. 5 depicts a process for deriving cumulative absorbed dose for the liver across three cycles of treatment

FIG. 6 depicts two variations of several on AI model input parameters used as training input variables and as inputs at the time of model inference. The left shows the absence of any post-injection scans used as inputs. The right shows a reduced number of post-injection scans than the full number of post-injection scans that are necessary for stand-alone dosimetry workflows.

FIG. 7 depicts a training phase (top) and live deployment phase (bottom) for the absorbed dose map method

FIG. 8 depicts the training phase (top) and live deployment phase (bottom) for the post-injection scan generator method

FIG. 9 depicts the training phase (top) and live deployment phase (bottom) for the absorbed dose value generator method

FIG. 10 depicts the training phase (top) and live deployment phase (bottom) for the post-injection value generator method

FIG. 11 depicts a process for iterating a specific treatment cycle using the Absorbed Dose Map Generator to achieve optimized levels of absorbed doses in targeted tumors and organs at risk

FIG. 12 depicts the use of actual ADMs produced from a stand-alone dosimetry workflow or generated ADMs from the ADMG or PISG methods as inputs to an AI model that predicts PET scans at future dates during (top) or following (bottom) a regimen of treatment cycles. Tumor progression can be observed from 6 to 9 months with death following at 10 months (bottom).

FIG. 13 depicts outputs from the ADMG (or PISG) method feeding forward into the Prognostic PET Generator model which feeds its output into the ADMG for the 2nd cycle of treatment and continues in this manner over the entire lifetime for a patient

FIG. 14 depicts input variables fed into a prognostic AI model to predict a variety of prognostic outcomes

FIG. 15 depicts input variables fed into an AI model used to predict the likelihood of occurrence for an adverse event arising during or following a regimen of treatment cycles

FIG. 16 depicts a process for optimizing the administered dose in the Nth cycle of a treatment regimen to achieve desirable prognostic outcomes and acceptable likelihood of adverse events occurrence

FIG. 17 depicts a process for optimizing the use of compound treatment agents in the Nth cycle of a treatment regimen to achieve desirable outcomes.

FIG. 18: An illustration of the proposed architecture and the multimodal training strategy. The top diagram uses a neural network to generate predicted values as well using a transformer model to generate predicted images while the bottom diagram shows just a transformer model for generating predicted images.

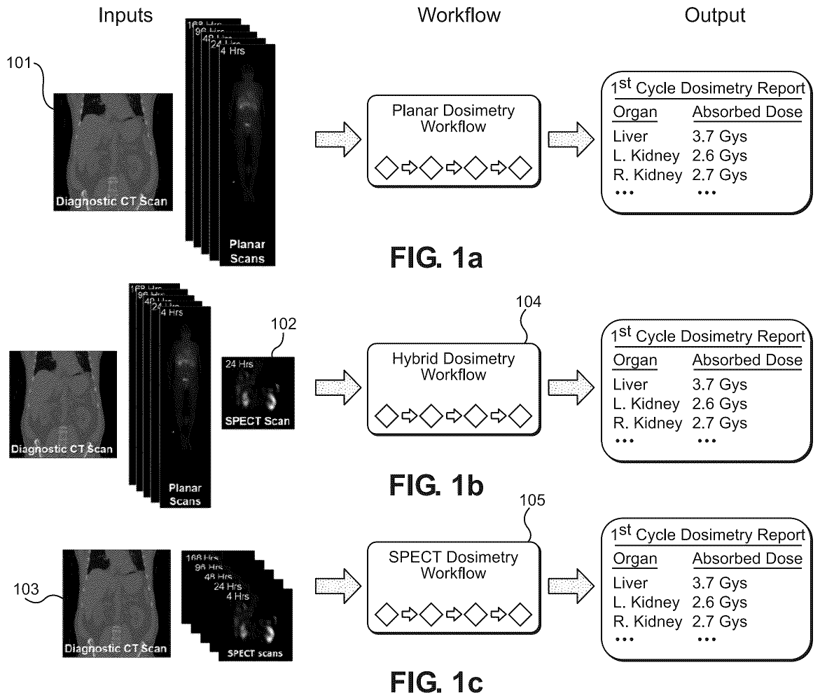

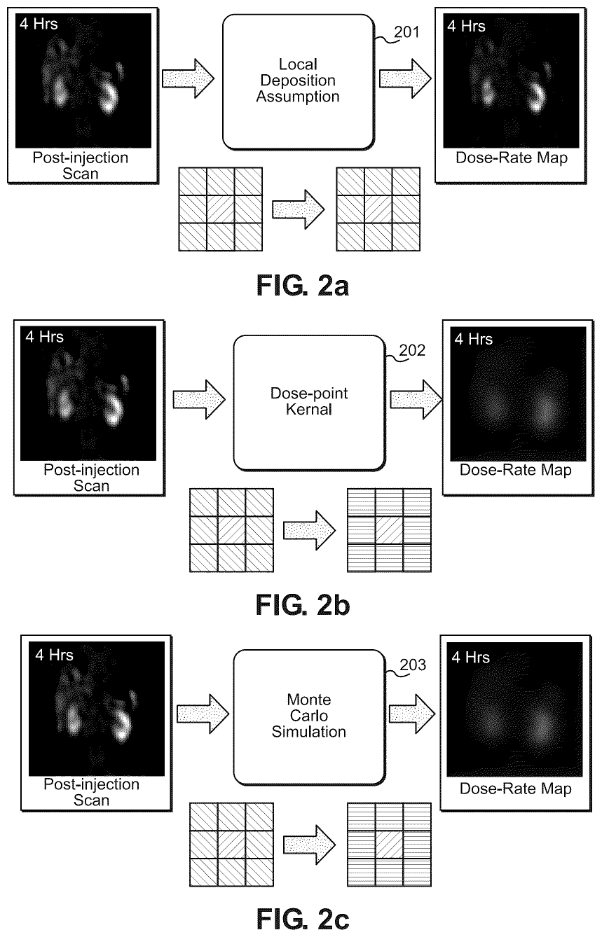

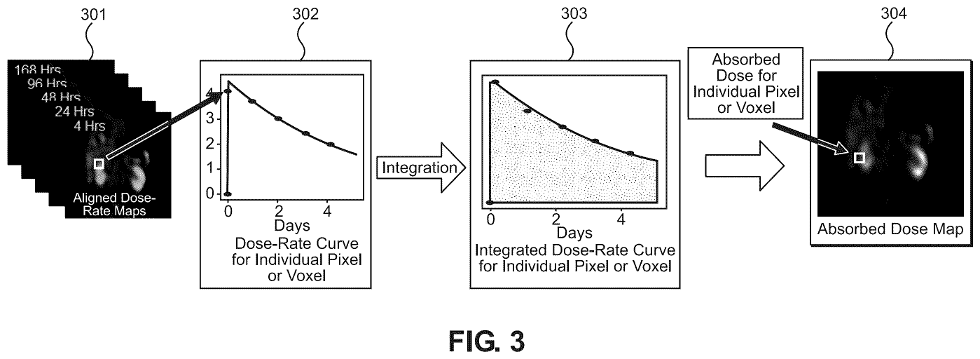

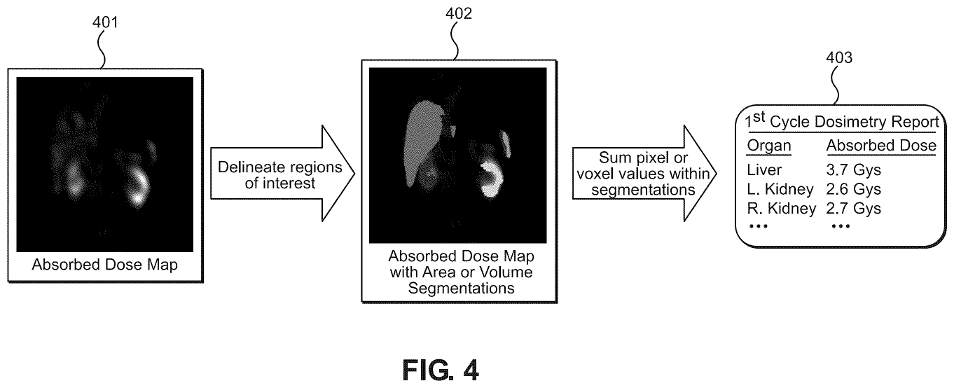

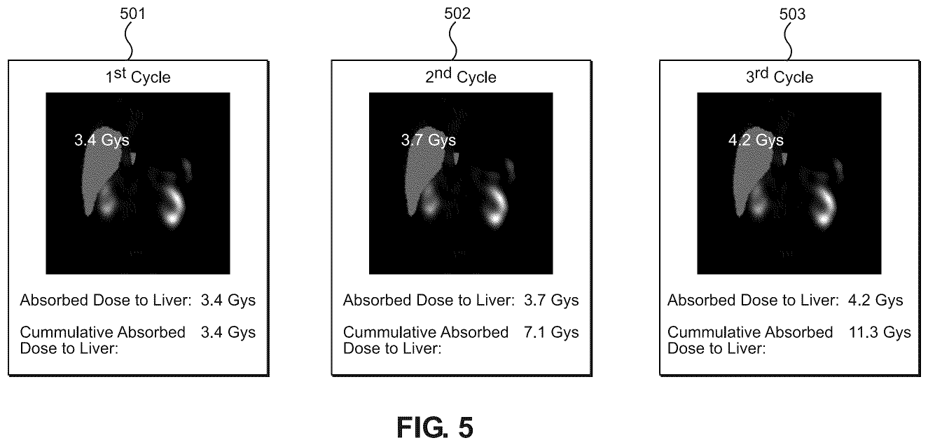

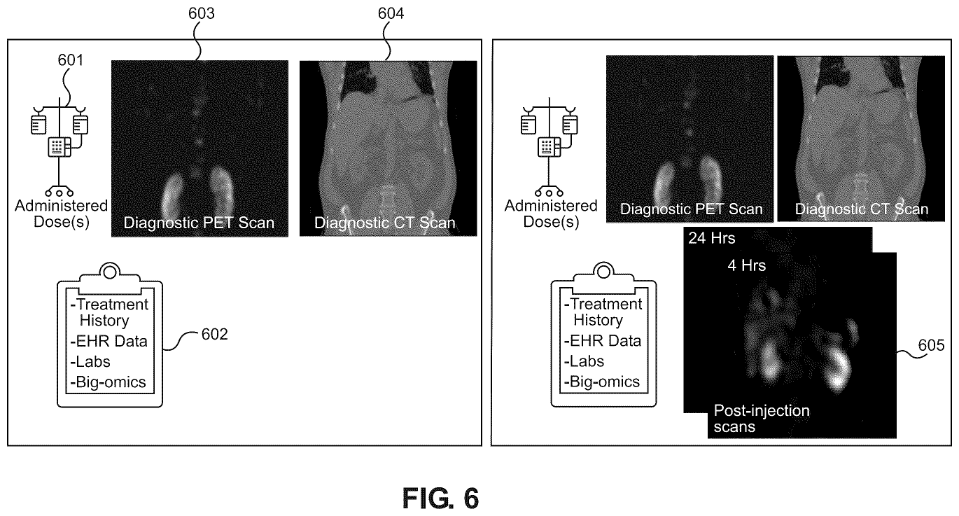

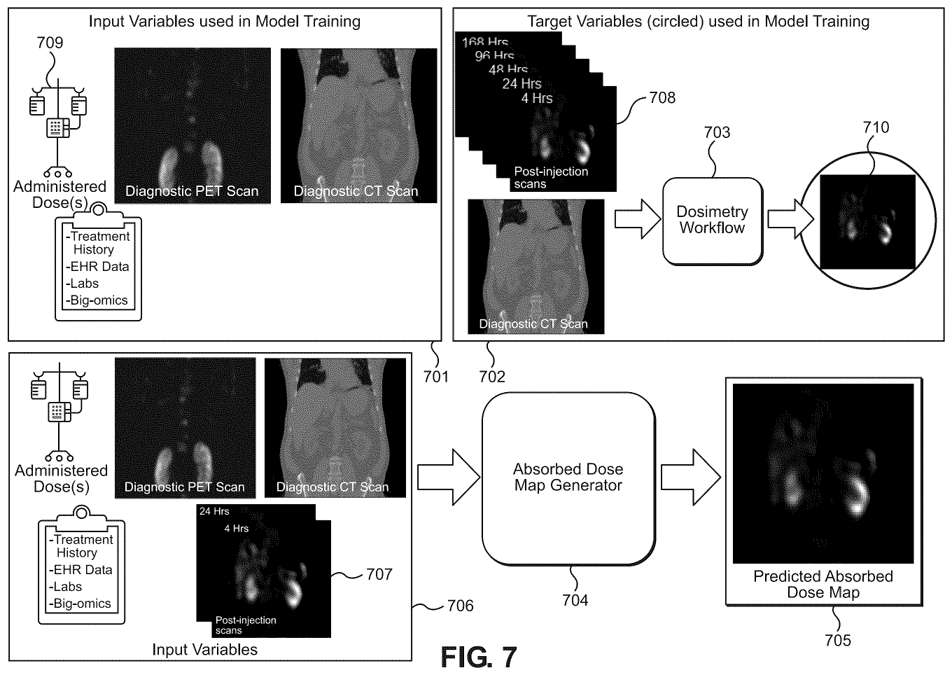

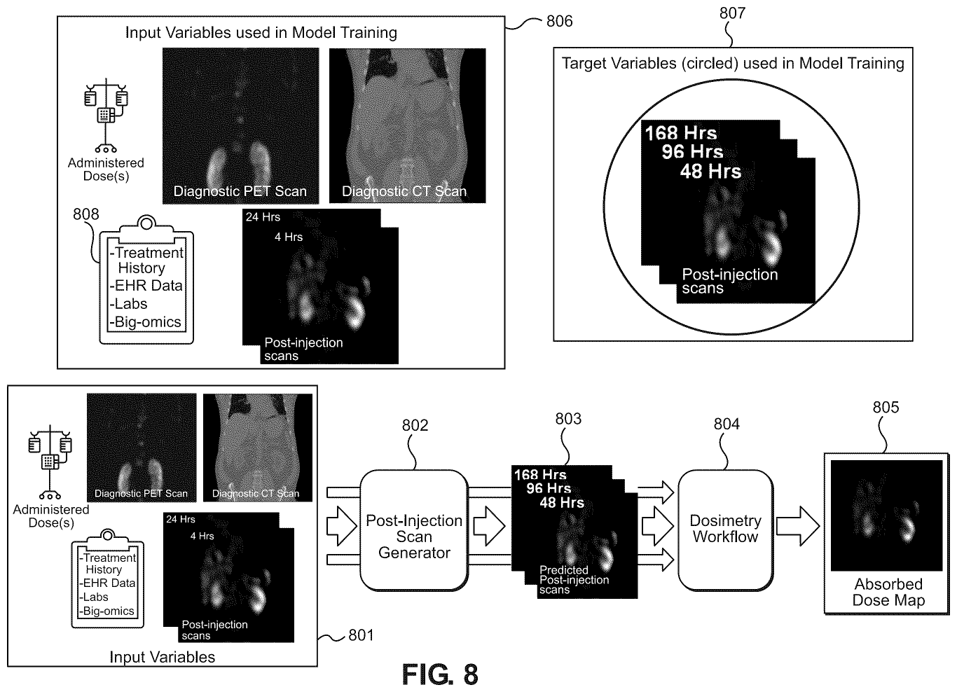

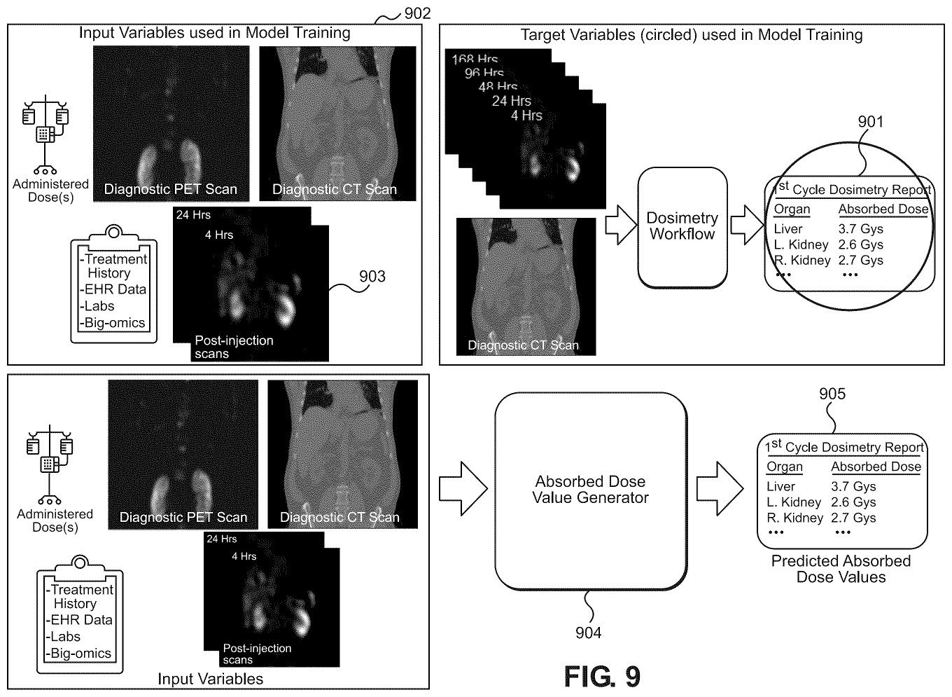

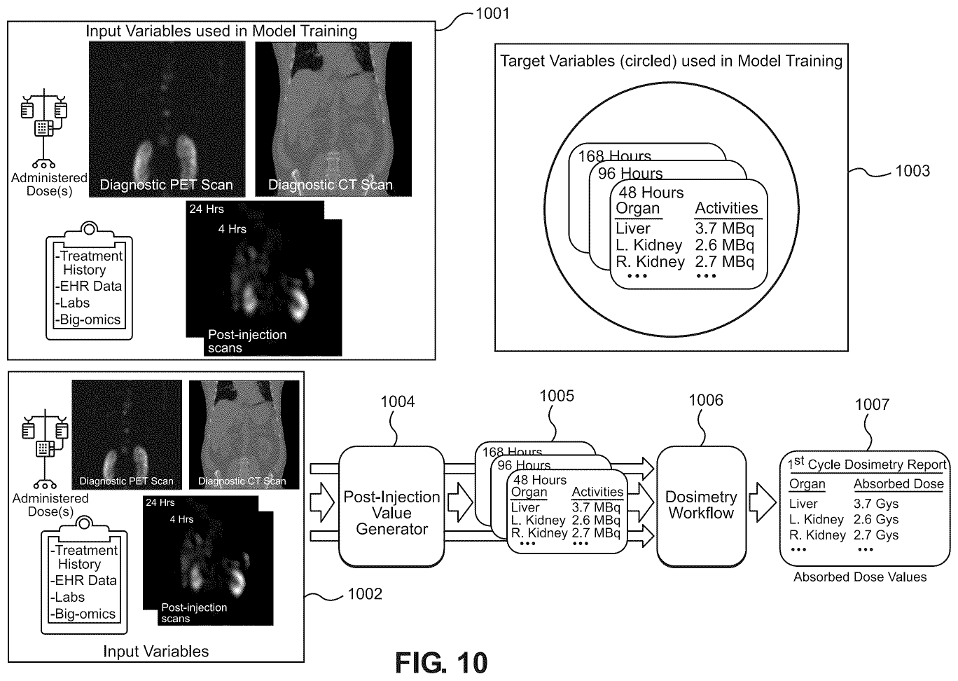

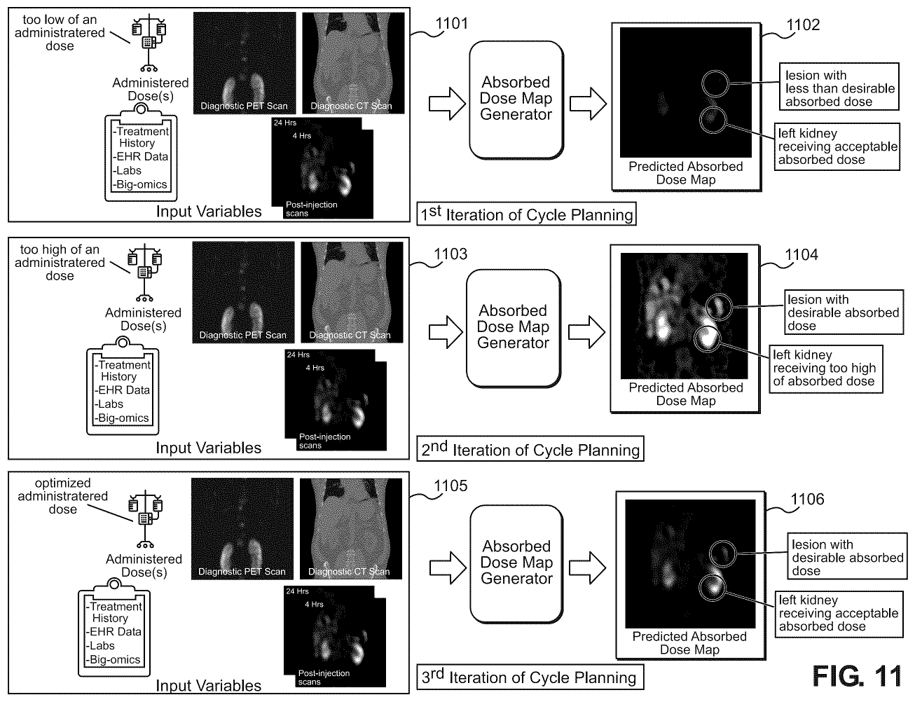

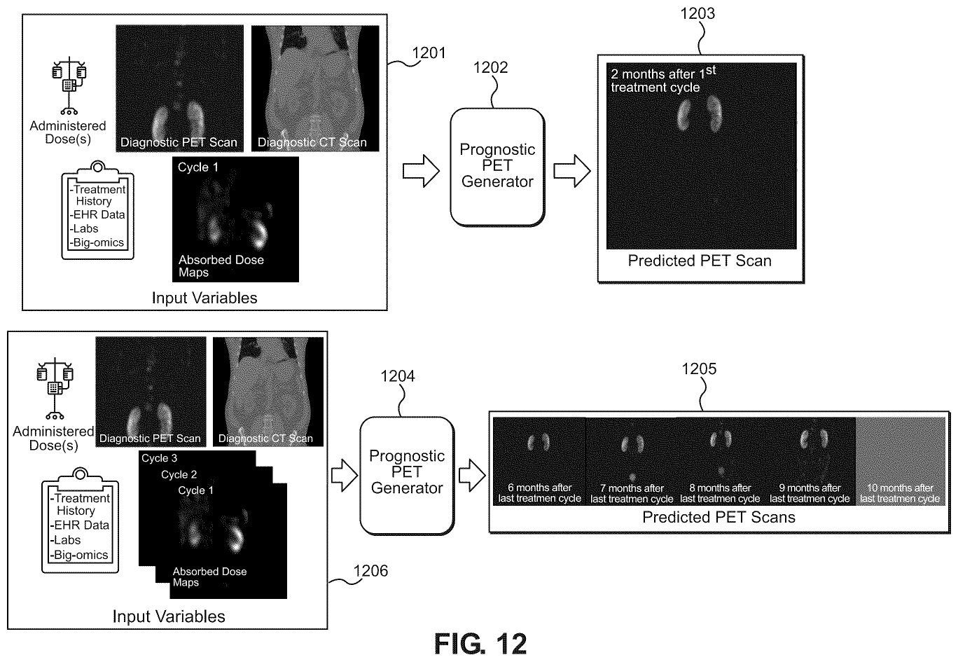

### DETAILED DESCRIPTION

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other features not described in detail herein. Additionally, some well-known structures or functions may not be shown or described in detail below, so as to avoid unnecessarily obscuring the relevant description. The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

Two embodiments of a machine learning method that works with a dosimetry workflow can be used to generate an Absorbed Dose Map (ADM) image data structure that may be stored as an image data file, tagged image data file or other data structure that stores pixel or voxel data along with location or other relevant data for the image. Each pixel or voxel may have a pixel value representing the amount of radiation dosage absorbed, such that when the image data is displayed on a computer screen, the brighter areas indicate higher absorption of radiation than the darker areas. In the first embodiment, termed the Absorbed Dose Map Generator (ADMG) method, the invention directly predicts the ADM data from the inputted PHD and IS data at the time of model deployment in the clinic. A training set for the ADMG model would use the base knowledge of known PHD and corresponding IS image data as the independent variables during model training (701). In a typical training exercise, the base knowledge set of hundreds or even thousands of patients' health data and corresponding scans may be used to train the machine learning engine. A training set for the ADMG model would use ADMs generated from a dosimetry workflow as the target variables during model training (702). Each data point used as a target variable in the ADMG model training therefore uses the full number of PIS required in a stand-alone dosimetry workflow. However, once the machine learning engine is trained, the dosimetry workflow (703) would not be used to generate an ADM when the ADMG method is live deployed. Rather, the machine learning engine Absorbed Dose Map Generator (704) generates a predicted ADM (705) using the patient IS and PHD as input variables (706). This reduces the total required computational time and power when the method is implemented in the clinic.

In the second embodiment, termed the post-injection scan generator (PISG) method, the machine learning model would generate the predicted PIS (803) at the time of model deployment and then feed these generated scans into the dosimetry workflow to obtain the ADM from the dosimetry workflow. As is the case with the ADMG method, the PISG module (802) would use PHD and IS as data inputs at the time of live deployment in the clinic (801). A training set for the PISG (806) would use base knowledge of known PHD and corresponding IS scan data as the independent input variables and target variable PIS (807) during model training, but unlike the ADMG, would not require a dosimetry workflow ADM output as the target variable for the training portion of the model's lifecycle. Instead, the base-knowledge PIS data corresponding to the based knowledge PHD and IS would be used (808). The principal advantage of the PISG method over the ADMG is that only one PIS per cycle need be acquired to develop a training set, so long as that one PIS is acquired at randomly selected timepoint of all of the possible post-injection timepoints that are necessary for the stand-alone dosimetry workflow. Using additionally acquired PIS beyond more than the one randomly acquired timepoint as supplemental inputs for model training improves the PISG's accuracy. A third embodiment of the invention is to include in the base knowledge training set other treatment parameters, for example, dosage rate values rather than predicted images. In addition, other non-image parameters may be predicted, including volume reduction of the tumor, the metabolic reduction of the tumor, and the presence or absence of an adverse reaction to the treatment. In this embodiment, the training set target variable are the same parameters corresponding to the known patient data. Once trained, the machine learning engine can then predict these parameters.

In one embodiment, for generating an absorption dosage map image data after application of the treatment, the invention takes as input a value representing the administered dose of the radiopharmaceutical (601), data representing patient treatment history and other EHR data (602), a diagnostic PET scan image (603) and a diagnostic CT scan image. (604). In one embodiment the value representing the administered dose is in the form of the count rate or radioactivity of the dose of the radiopharmaceutical injected into the patient, for example in units called becqerel (601). In another embodiment, the administered dose value can be hypothetical in that the dose value can be tested by the system by using the system to predict the effect of such a dosage both in efficacy and side effects before that dosage is actually applied to the patient (605). Optionally, one or more post-injection scans and patient data (707) are used as inputs into the machine learning model engine. The output is a predicted absorbed dose map image (705). In order to train the machine learning engine, a series of post injection scans (708) for a set of patients are input as well as the corresponding patient IS diagnostic scans and PHD patient data (709) and the internal parameters that define the machine learning engine function adjusted so that the machine output is a predicted ADM image (710) that the sufficiently matches a known absorption map output image from the dosimetry workflow for the corresponding patient or a known PIS image data indicating the amount of radiation being emitted from the tissue as a result of the application of the radiopharmaceutical for the corresponding patient. As this training is done over tens, hundreds or thousands of patient histories and corresponding patient scans, the trained machine learning engine improves its accuracy of prediction. Together, the trained machine learning engine and components are an Absorbed Dose Map Generator. (704). The process may be enhanced by using a machine learning engine to take the administered dosage, diagnostic PET scan, diagnostic CT scan, one or more post-injection scans and patient data inputs (801) to generate predicted post-injection scan images. (803) These predicted PIS may be used as input into an existing dosimetry workflow. (804).

In one embodiment the machine learning engine that generates the predicted Absorbed Dosage Map image can be replaced with a machine learning engine that directly generates predicted absorbed dosage values (901) from the fewer actual PIS and other patient data as input (902) using a machine learning engine trained using a set of PIS images (903). In lieu of predicting entire dose maps in the case of the ADMG, specific AD values for organs or tumors could be predicted for an area or volume using an absorbed dose value generator (ADVG) model. In the lieu of predicting entire PIS in the case of the PISG, specific activity values or dose-rate values could be predicted for an area or volume using a post-injection value generator (PIVG) which would then be inputted into a dosimetry workflow. Predicting single values for an area or volume (905) instead of predicting entire maps or PIS would require simpler model complexity; however, the same input variables as those used in the ADMG or PISG would be used as the input variables for ADVG or PIVG models (904)(1004). In this embodiment, the machine learning engine architecture is not necessarily a neural network or deep learning network. Instead, the machine learning engine may be a support vector machine (SVM) or perform linear regression where coefficients of the linear regression are adjusted during the training process. In one embodiment, the overlay of organ segmentations may be used to select for absorbed dose values for those organ regions and using only those selected values for those regions as input into the SVM. Further, a survival forest architecture or deep learning network may be used for generating a predicted parameter output representing adverse event or other treatment result quality parameters.

In another embodiment the predicted dose rate values (1005) or predicted detected emissions can be generated for a series of times after the dose was administered whereby the administered dosage, diagnostic PET scan, diagnostic CT scan, one or more post-injection scans and patient data inputs (1002) to generate predicted post-injection absorption values for the segmented organ regions (1005). A series of known dose rate values or detected emissions (1003) may be used as the target values to train the post-injection value generator engine (1004). The series of dose rate values or detected emissions may be used as input into a dosimetry workflow (1006) to output the predicted absorbed dose for a given cycle that the administered dose was part of (1007).

Attending physicians can use the ADMG, PISG, ADVG and PIVG in treatment planning of a specific cycle to ensure that desirable levels of absorbed doses are absorbed by specific tumors and treating physicians could also use these methods to lower administered doses to organs at risk. The image output of the absorbed does map generator may be used to evaluate dosages. Where a dosage is too low (1101), the map would show modest absorption (1102). Where the dosage is too high, (1103) the output image would show excessive absorption (1104). Where the dosage is optimized, ((1105) the output image would present an absorbed dose map with absorption values within recommended dose limits for organs at risk with the maximum absorption values for tumours (1106).

The absorbed dose map generator may be used to create a predicted absorption dose map image for the scan area. In this embodiment, the administered dosage, diagnostic PET scan, diagnostic CT scan, one or more absorption dose maps and patient data inputs (1201) are used to train a machine learning engine (1202) to generate a predicted absorbed dose map scan image. (1203). As dosages are applied in subsequent cycles of treatment, a post treatment PET prediction engine (1204) can then generate predicted PET scan outputs (1205) for the subsequent series of cycles as a function of time using the predicted absorbed dose map image (1206) as input.

Once a predicted absorption dose map is generated, (1302), it can be used to generate the predicted PET scan (1303) and that fed back into the absorption dose map generator (1304) with the next cycle dosage, CT scan and the patient data in order to generate a predicted PET scan for the next cycle of treatment. This feedback can be conducted over and over to predict the results of the next cycle of treatment. This approach permits the clinician to evaluate a series of treatments of the radio-pharmaceutical by predicting the result of a series of treatments on the same patient.

The process may be used to take the series of absorbed dose maps and other input data for the patient (1401) to train a machine learning engine (1402) to generate a prognostic value output (1403) that indicates the quality of result. In the most simple version, the output is Boolean value indicating whether the outcome of the cycle of treatments is acceptable or not. In one embodiment, the machine learning engine (1502) operating as an adverse event predictor can use the patient IS and PHD information (1501) to generate a predictive value that indicates the likelihood of an adverse event arising from the treatment plan. (1503).

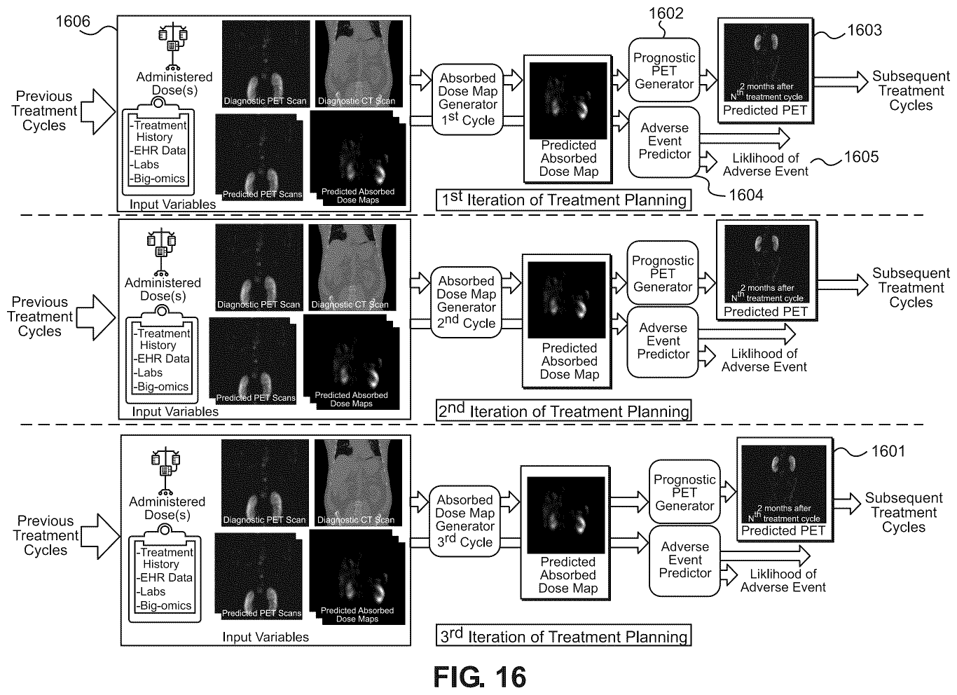

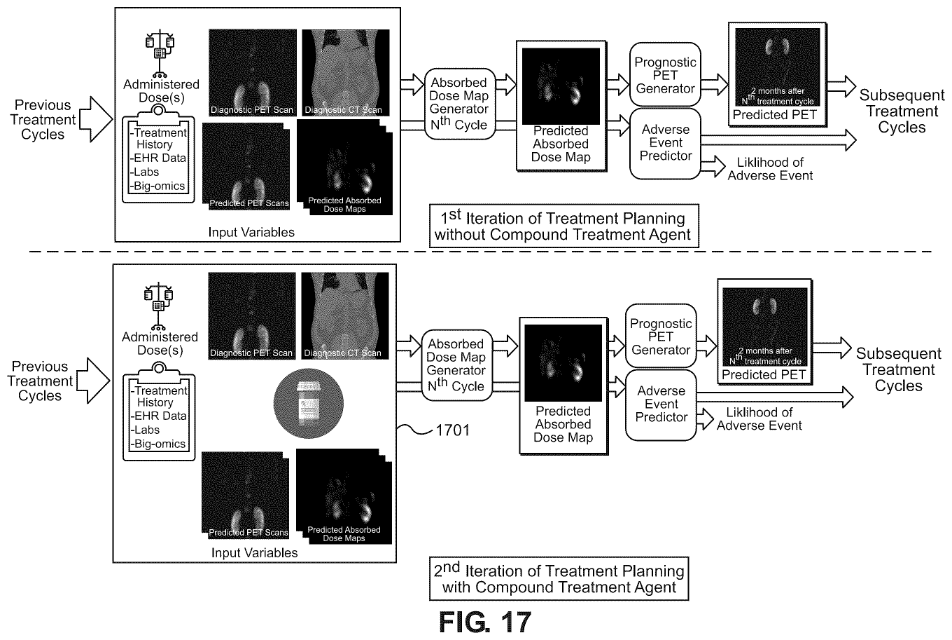

The processes may be combined to create an iterative treatment plan evaluation process as shown in FIG. 16. This process would use the absorbed dose map generator to generate for n cycles of treatment, the predicted absorption map if those cycles were applied. (1601). The predicted PET scan generator (1602) would then create a PET scan image output (1603) and the adverse event predictor engine (1604) would generate a value representing the likelihood of an adverse event for that treatment plan (1605). The administered dosages as input (1606) can be optimized before the actual administration of the radiation to be a value that generates an acceptable PET scan prediction (1603) as well as an acceptable likelihood of an adverse event (1605).

In yet another embodiment, the iterative process of planning a series of treatment cycles can be used to determine a prescription for using a compound treatment agent (1701).

The main advantage of using predicted maps or predicted PIS over generated values is that pixel or voxel-level information from the outputted ADM from either the ADMG or PISG can be fed forward into other machine learning models trained to predict the likelihood of adverse events, or tumor response at the voxel or pixel level relying on imagery as input. For example, PHD in combination with ADMs could be used as input variables to predict post-treatment PETs at a future point(s) in time during or following a regimen of treatment cycles. Tumors clearly visible in a predicted PET, at for example 9 months, may not be visible or immediately noticeable in an actual PET at 6 months and therefore a prognostic PET generator may aid radiologists in early detection of recurrent tumors or even new metastases. The predicted PETs can then be fed forward into an ADMG or PISG, possibly specific for a given cycle of treatment, to produce ADMs in future treatment cycles. The combination of using such models in a linear feed forward manner like this can then be used for treatment planning and prognosis over the entire lifetime for a patient. Each treatment phase generates data for the machine learning engine model corresponding to that phase, so with multiple phase treatment, there is multiple model training data. The output of each phase of treatment can be used to drive the training of the machine learning engine model that corresponding to the next phase of treatment.

Machine learning models for treatment result prediction need not necessarily predict PET scan images but could also be used to predict numerical values that re-present the quality of the treatment outcome. Common prognostic values include time-to-event metrics like overall survival and progression free survival but can also include the likelihood of surviving beyond a certain period of time. Predicted values could also include specific lab values germane to disease outcome and might include prostate-specific antigen and chromogranin levels.

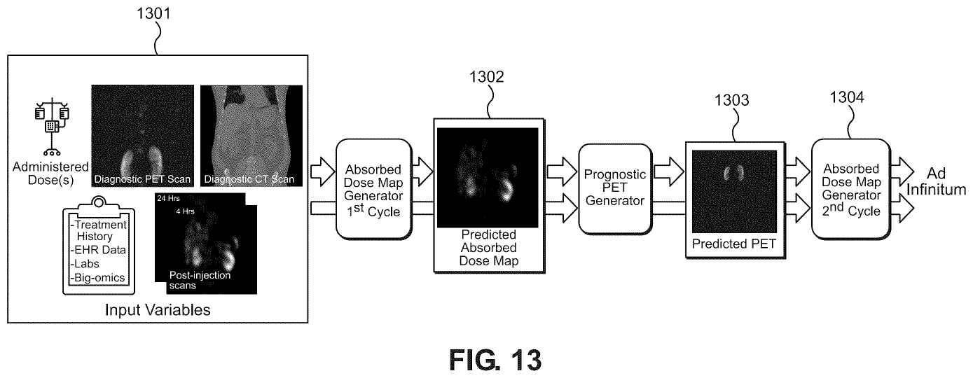

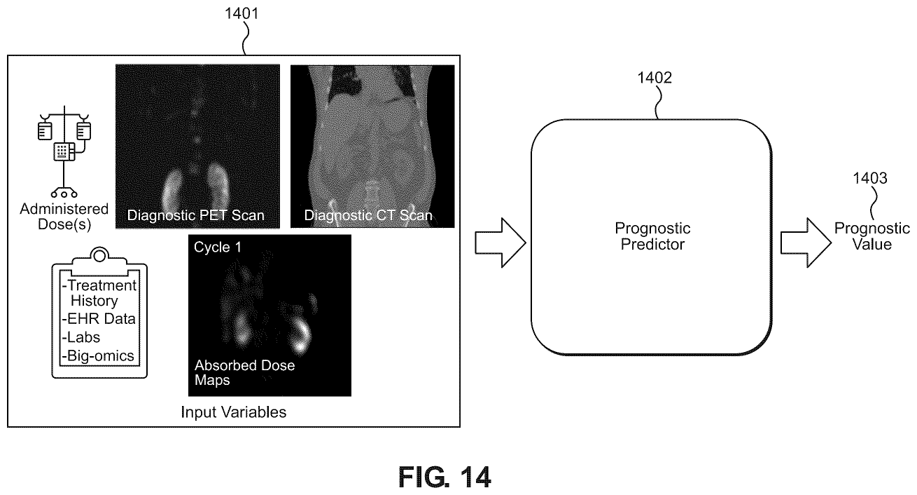

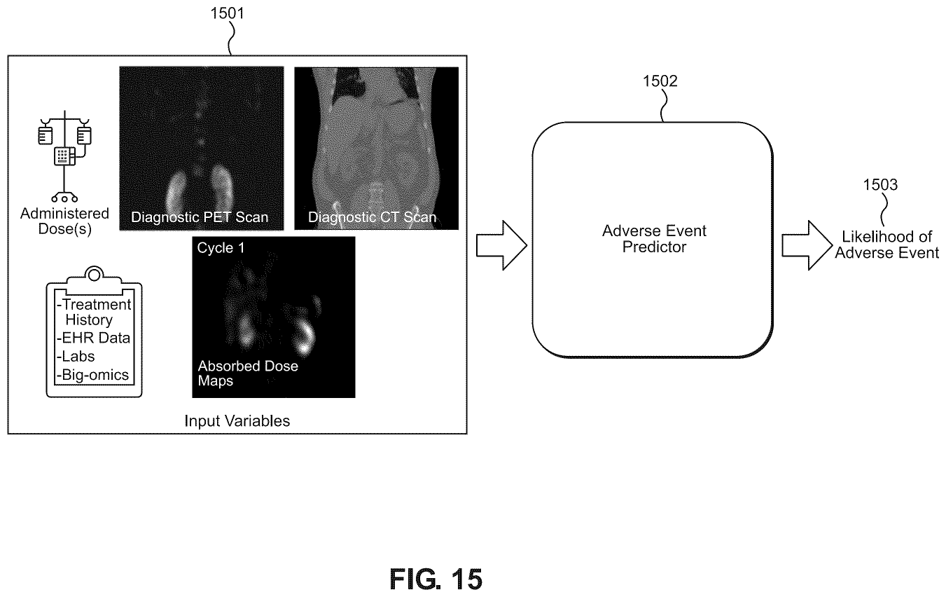

Machine learning engines can also be trained to predict the likelihood of adverse events arising over the course of a patient's treatments that occur with limited frequency in SIRT and can include but are not limited to anemia, diarrhea, elevated ALT levels, elevated AST levels, fatigue, leukopenia, nausea, nephrotoxicity, thrombocytopenia and xerostomia. Combining the outputs of these different types of machine learning methods together in a feedforward manner like that shown in FIG. 13 then allows an attending physician to optimize specific cycles of treatment plans over an entire regimen of treatment cycles by adjusting the inputted administered dose. Similarly, to adjusting the inputted administered doses, attending physicians can also adjust the addition or removal of a compound therapy treatments with other cancer fighting drugs.

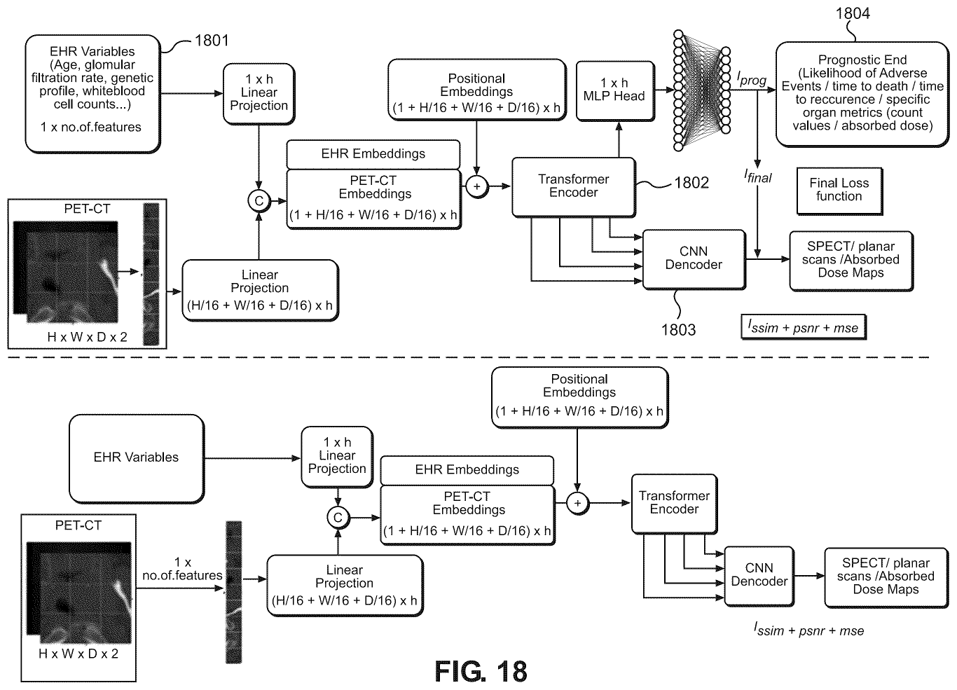

The claimed invention automates the process by using a machine learning engine to analyze the patient's images in order to conduct organ and tumor segmentation, tumor tracking and analysis and prediction calculations automatically. The machine learning engine (FIG. 18) may be comprised of a neural network, preferably a neural network that is specifically trained using pre treatment and post treatment image data and electronic health record data from a population of patients and a set of correct organ and tumor segmentations. Different parts of the system may use different machine learning models. For example, a deep learning model for ADM prediction, a survival forest for prognostic prediction and a separate deep learning model for adverse event likelihood prediction.

Analyzing the image data and the EHR data together using the machine learning engine takes several steps:
a. First, the image is broken up into a series of patches. For example, a 1024×1024 pixel image can be transformed into a 4096×16×16 data structure, where each 16×16 element is a patch.
b. Then, the 16×16 array can be projected linearly to create a 4096×256 image embedding data structure.
c. third, the EHR data can be represented by a vector. For example, a 12 variable EHR vector may be linearly projected out to a 256 element vector to be the EHR embedding.
d. fourth, the system will concatenate image and EHR embedding to get final embedding matrix, in the example the final embedding is a 4097×256 matrix.
e. The next step is to incorporate the positional information into the final embedding matrix. The positional embedding matrix is preferably the same dimensions as the final embedding matrix from step d. In the preferred embodiment, the positional embedding matrix is added to the final embedding matrix c.
f. The output of step e, which is the Final embedding matrix+Positional embedding matrix is input into a transformer machine learning architecture.
g. The output of the transformer is used to drive a CNN (convolution neural network) decoder to produce a predicted image. Further, the output of the transformer may be used to feed a MLP (multi level perceptron) head to output one or more predicted treatment result parameters.

In one embodiment, the positional information of the patches is an encoding of the relational position of the patch for the position encoding matrix elements. Other embodiments can rely on absolute positional embedding for an element in the matrix, which is deterministic based on the patch position. In the preferred embodiment, the relational embedding can include the use of learnable positional encoding techniques to improve the quality of the positional embedding matrix. In this embodiment, the relational embedding matrix is comprised of a set of matrix elements whose values are determined through the training process of the machine learning engine. This training is performed as part of the training of the entire system. The determination of the relational embedding matrix is part of the results of executing a back propagation process when training the system. The matrix values are adjusted to minimize the loss function in accordance with back propagation algorithmic procedures.

The system architecture linearly projects EHR (1801) and multimodal images into a feature vector and feeds it into a Transformer encoder. (1802) The CNN decoder (1803) is fed with the input images, skip connection outputs at different layers, and the final layer output to perform the segmentation, whereas the prognostic end (1804) utilizes the output of the last layer of the encoder to predict the risk score.

Transformer Encoder: A transformer encoder is utilized to attend multimodal imaging and EHR data to synthesize scans (SPECT/planar/PET) and prognostic outcomes by projecting multimodal data to same embedding space. In the preferred embodiment, input images are split into small patches, typically 16×16 pixel patches. Each patch has a position parameter that indicates its location in the original image. The EHR is a vector of length R×i, where i is the number of EHR parameters. An advantage of this network is that the encoder itself embeds both the CT/PET and EHR data and encodes positions for them accordingly while extracting dependencies (i.e. attention) between the different modalities. 3D image with dimensions x∈R\hat{ }(H×W×D×C) is reshaped into a sequence of flattened 2D patches xp∈R\hat{ }(n [symbol] ×(p [symbol] \hat{ }3c)), where H, W, and D are the height, width, and depth of the 3D image respectively, C denotes the number of channels, P×P×P represents each patch's dimensions, and n=HWD/P\hat{ }3 is the number of patches extracted. These patches are then projected to the embedding dimension h, forming a matrix I∈R\hat{ }(n×h).

Simultaneously, EHR data is also projected to a dimension E∈R\hat{ }(1×h). Both projections of images and EHR are concatenated, forming a matrix X∈R\hat{ }((n+1)×h). Positional encodings with the same dimension are added to each of the patches and the EHR projection as learnable parameters. The class token is dropped from the ViT as this embodiment does not address a classification task. The resulting embeddings are fed to a transformer encoder consisting of 12 layers, following the same pipeline as the original ViT, with normalization, multi-head attention, and multi-layer perceptron. The purpose of using self-attention is to learn relations between n+1 number of embeddings, including images and EHR. (n is number of patches)

Image Synthesis End: Image synthesis end is a CNN decoder. Original images are fed to the decoder along with skip layer from transformers.

Prognostic End: Prognostic path receives input from the final layer of the transformer with dimension R\hat{ }((n+1)×h). The mean value of the input is computed, reducing the dimensions to R\hat{ }(1×h). This latent vector is then forwarded to the multilayer perceptron to predict prognostic values.

Loss Functions: Appropriate loss functions and their combinations will be used for the image synthesis and prognostic predictions. For example, combination structural similarity index, PSNR, mean squared error and blurred loss will be used for image synthesis while negative loss likelihood will be used for prognostic predictions.

The system and method automates the generation of a therapeutic dosage recommendation and a predicted PET scan, which is indicative of the effectiveness of the therapy. Alternatively, rather than a predicted PET scan, the system can generate a predicted metric indicating the quality or efficacy of the treatment plan. In one example, the metric may be the tumor volume reduction factor. In this embodiment, other machine learning models than a neural network may be used, for example, support vector machines (SVM). The output of such an SVM can be tumour reduction percentage or other factor, or a number of Greys absorbed by the tumor.

Other metrics indicating the quality or efficacy of the treatment result may be used. For example, the amount of targeting agent that binds to a tumor, for example PSMA-617 or PSMA INT may be considered parameters that may be predicted using the trained machine learning engine. PSMA is one receptor on the tumor cell surface that binds to the targeting agent that is molecularly bound to the injected radioactive marker. Training the machine learning engine with PHD, IS and measured PSMA-617 or PSMA INT concentrations after treatment would then make the engine predict the expected PSMA concentration response after treatment.

Other parameters that may be metrics indicating the quality of the treatment include blood levels of certain biochemical markers that are correlated with tumor activity, for example, PSA concentrations in the blood stream arising from a prostate cancer. Another example is evaluating the strength of the tumor metabolism. For example, using the FDG as a metabolism indicator arising from its heightened uptake by tumor cells in comparison to normal cells and its radioactivity being detected by a PET scan. In some embodiments, the techniques permit using the predicted absorbed dosage map and predicted adverse event to determine a treatment plan without running a SPECT scan at all.

In this embodiment the system can predict the value of a treatment outcome parameter corresponding to a patient by:
Receiving a PHD data set corresponding to the patient;
Receiving an IS data set corresponding to the patient;
Receiving an administered dose value corresponding to the patient;
Inputting the received PHD data set, received IS data set and administered dose value into a machine learning engine trained using known PHD, corresponding known IS data, corresponding known administered dose values as inputs and corresponding known predicted treatment outcome parameter values as an output target variable; and
Using the trained machine learning engine to determine a predicted value of such treatment outcome parameter. The treatment outcome parameter may be one of a numerical time-to-event metric, a probability, a risk score, a survival interval or a progression free survival interval. Similarly, the treatment outcome parameter may be one of a numerical time-to-event metric value comprised of at least one of prostate-specific antigen and chromogranin levels. The treatment outcome parameter may be a probability value of an adverse event. The adverse event value may be the predicted occurrence or probability of occurrences of one of anemia, diarrhea, constipation, fatigue, myelosuppression (i.e., decreased hemoglobin, decreased platelets, decreased leukocytes, and decreased neutrophils), spontaneous and/or uncontrolled episodes of bleeding, infection, sepsis, acute kidney injury and/or severe renal toxicity, electrolyte abnormalities, pain, vertigo, temporary or permanent infertility, embryo-fetal toxicity, leukopenia, nausea, vomiting, hemotoxicity, nephrotoxicity, thrombocytopenia, dry eye, xerophthalmia, dry mouth, xerostomia and secondary malignancy.

### Operating Environment:

The system is typically comprised of a central server that is connected by a data network to a user's computer. The central server may be comprised of one or more computers connected to one or more mass storage devices. The precise architecture of the central server does not limit the claimed invention. Further, the user's computer may be a laptop or desktop type of personal computer. It can also be a cell phone, smart phone or other handheld device, including a tablet. The precise form factor of the user's computer does not limit the claimed invention. Examples of well-known computing systems, environments, and/or configurations that may be suitable for use with the invention include, but are not limited to, personal computers, server computers, handheld computers, laptop or mobile computer or communications devices such as cell phones, smart phones, and PDA's, multiprocessor systems, microprocessor-based systems, set top boxes, programmable consumer electronics, network PCs, minicomputers, mainframe computers, distributed computing environments that include any of the above systems or devices, and the like. Indeed, the terms “computer,” “server,” and the like may be used interchangeably herein, and may refer to any of the above devices and systems.

The user environment may be housed in the central server or operatively connected to it remotely using a network. In one embodiment, the user's computer is omitted, and instead an equivalent computing functionality is provided that works on a server. In this case, a user would log into the server from another computer over a network and access the system through a user environment, and thereby access the functionality that would in other embodiments, operate on the user's computer. Further, the user may receive from and transmit data to the central server by means of the Internet, whereby the user accesses an account using an Internet web-browser and browser displays an interactive web page operatively connected to the central server. The server transmits and receives data in response to data and commands transmitted from the browser in response to the customer's actuation of the browser user interface. Some steps of the invention may be performed on the user's computer and interim results transmitted to a server. These interim results may be processed at the server and final results passed back to the user.

The Internet is a computer network that permits customers operating a personal computer to interact with computer servers located remotely and to view content that is delivered from the servers to the personal computer as data files over the network. In one kind of protocol, the servers present webpages that are rendered on the customer's personal computer using a local program known as a browser. The browser receives one or more data files from the server that are displayed on the customer's personal computer screen. The browser seeks those data files from a specific address, which is represented by an alphanumeric string called a Universal Resource Locator (URL). However, the webpage may contain components that are downloaded from a variety of URL's or IP addresses. A website is a collection of related URL's, typically all sharing the same root address or under the control of some entity. In one embodiment different regions of the simulated space displayed by the browser have different URL's. That is, the webpage encoding the simulated space can be a unitary data structure, but different URL's reference different locations in the data structure. The user computer can operate a program that receives from a remote server a data file that is passed to a program that interprets the data in the data file and commands the display device to present particular text, images, video, audio and other objects. In some embodiments, the remote server delivers a data file that is comprised of computer code that the browser program interprets, for example, scripts. The program can detect the relative location of the cursor when the mouse button is actuated, and interpret a command to be executed based on location on the indicated relative location on the display when the button was pressed. The data file may be an HTML document, the program a web-browser program and the command a hyper-link that causes the browser to request a new HTML document from another remote data network address location. The HTML can also have references that result in other code modules being called up and executed, for example, Flash or other native code.

The invention may also be entirely executed on one or more servers. A server may be a computer comprised of a central processing unit with a mass storage device and a network connection. In addition a server can include multiple of such computers connected together with a data network or other data transfer connection, or, multiple computers on a network with network accessed storage, in a manner that provides such functionality as a group. Practitioners of ordinary skill will recognize that functions that are accomplished on one server may be partitioned and accomplished on multiple servers that are operatively connected by a computer network by means of appropriate inter process communication. In one embodiment, a user's computer can run an application that causes the user's computer to transmit a stream of one or more data packets across a data network to a second computer, referred to here as a server. The server, in turn, may be connected to one or more mass data storage devices where the database is stored. In addition, the access of the website can be by means of an Internet browser accessing a secure or public page or by means of a client program running on a local computer that is connected over a computer network to the server. A data message and data upload or download can be delivered over the Internet using typical protocols, including TCP/IP, HTTP, TCP, UDP, SMTP, RPC, FTP or other kinds of data communication protocols that permit processes running on two respective remote computers to exchange information by means of digital network communication. As a result a data message can be one or more data packets transmitted from or received by a computer containing a destination network address, a destination process or application identifier, and data values that can be parsed at the destination computer located at the destination network address by the destination application in order that the relevant data values are extracted and used by the destination application. The precise architecture of the central server does not limit the claimed invention. In addition, the data network may operate with several levels, such that the user's computer is connected through a fire wall to one server, which routes communications to another server that executes the disclosed methods.

The server can execute a program that receives the transmitted packet and interpret the transmitted data packets in order to extract database query information. The server can then execute the remaining steps of the invention by means of accessing the mass storage devices to derive the desired result of the query. Alternatively, the server can transmit the query information to another computer that is connected to the mass storage devices, and that computer can execute the invention to derive the desired result. The result can then be transmitted back to the user's computer by means of another stream of one or more data packets appropriately addressed to the user's computer. In addition, the user's computer may obtain data from the server that is considered a website, that is, a collection of data files that when retrieved by the user's computer and rendered by a program running on the user's computer, displays on the display screen of the user's computer text, images, video and in some cases outputs audio. The access of the website can be by means of a client program running on a local computer that is connected over a computer network accessing a secure or public page on the server using an Internet browser or by means of running a dedicated application that interacts with the server, sometimes referred to as an “app.” The data messages may comprise a data file that may be an HTML document (or other hypertext formatted document file), commands sent between the remote computer and the server and a web-browser program or app running on the remote computer that interacts with the data received from the server. The command can be a hyper-link that causes the browser to request a new HTML document from another remote data network address location. The HTML can also have references that result in other code modules being called up and executed, for example, Flash, scripts or other code. The HTML file may also have code embedded in the file that is executed by the client program as an interpreter, in one embodiment, Javascript. As a result a data message can be a data packet transmitted from or received by a computer containing a destination network address, a destination process or application identifier, and data values or program code that can be parsed at the destination computer located at the destination network address by the destination application in order that the relevant data values or program code are extracted and used by the destination application.

The invention may also be practiced in distributed computing environments where tasks are performed by remote processing devices that are linked through a communications network. In a distributed computing environment, program modules may be located in both local and remote computer storage media including memory storage devices. Practitioners of ordinary skill will recognize that the invention may be executed on one or more computer processors that are linked using a data network, including, for example, the Internet. In another embodiment, different steps of the process can be executed by one or more computers and storage devices geographically separated by connected by a data network in a manner so that they operate together to execute the process steps. In one embodiment, a user's computer can run an application that causes the user's computer to transmit a stream of one or more data packets across a data network to a second computer, referred to here as a server. The server, in turn, may be connected to one or more mass data storage devices where the database is stored. The server can execute a program that receives the transmitted packet and interpret the transmitted data packets in order to extract database query information. The server can then execute the remaining steps of the invention by means of accessing the mass storage devices to derive the desired result of the query. Alternatively, the server can transmit the query information to another computer that is connected to the mass storage devices, and that computer can execute the invention to derive the desired result. The result can then be transmitted back to the user's computer by means of another stream of one or more data packets appropriately addressed to the user's computer. In one embodiment, a relational database may be housed in one or more operatively connected servers operatively connected to computer memory, for example, disk drives. In yet another embodiment, the initialization of the relational database may be prepared on the set of servers and the interaction with the user's computer occur at a different place in the overall process.

The method described herein can be executed on a computer system, generally comprised of a central processing unit (CPU) that is operatively connected to a memory device, data input and output circuitry (TO) and computer data network communication circuitry. The computer system may also be comprised of a graphics processor or other tensor processing unit that assists the CPU in executing the computer processes that comprise a neural network architecture machine learning engine. Computer code executed by the CPU can take data received by the data communication circuitry and store it in the memory device. In addition, the CPU can take data from the I/O circuitry and store it in the memory device. Further, the CPU can take data from a memory device and output it through the IO circuitry or the data communication circuitry. The data stored in memory may be further recalled from the memory device, further processed or modified by the CPU in the manner described herein and restored in the same memory device or a different memory device operatively connected to the CPU including by means of the data network circuitry. In some embodiments, data stored in memory may be stored in the memory device, or an external mass data storage device like a disk drive. In yet other embodiments, the CPU may be running an operating system where storing a data set in memory is performed virtually, such that the data resides partially in a memory device and partially on the mass storage device. The CPU may perform logic comparisons of one or more of the data items stored in memory or in the cache memory of the CPU, or perform arithmetic operations on the data in order to make selections or determinations using such logical tests or arithmetic operations. The process flow may be altered as a result of such logical tests or arithmetic operations so as to select or determine the next step of a process. For example, the CPU may obtain two data values from memory and the logic in the CPU determine whether they are the same or not. Based on such Boolean logic result, the CPU then selects a first or a second location in memory as the location of the next step in the program execution. This type of program control flow may be used to program the CPU to determine data, or select a data from a set of data. The memory device can be any kind of data storage circuit or magnetic storage or optical device, including a hard disk, optical disk or solid state memory. The IO devices can include a display screen, loudspeakers, microphone and a movable mouse that indicate to the computer the relative location of a cursor position on the display and one or more buttons that can be actuated to indicate a command.

The computer can display on the display screen operatively connected to the I/O circuitry the appearance of a user interface. Various shapes, text and other graphical forms are displayed on the screen as a result of the computer generating data that causes the pixels comprising the display screen to take on various colors and shades or brightness. The user interface may also display a graphical object referred to in the art as a cursor. The object's location on the display indicates to the user a selection of another object on the screen. The cursor may be moved by the user by means of another device connected by I/O circuitry to the computer. This device detects certain physical motions of the user, for example, the position of the hand on a flat surface or the position of a finger on a flat surface. Such devices may be referred to in the art as a mouse or a track pad. In some embodiments, the display screen itself can act as a trackpad by sensing the presence and position of one or more fingers on the surface of the display screen. When the cursor is located over a graphical object that appears to be a button or switch, the user can actuate the button or switch by engaging a physical switch on the mouse or trackpad or computer device or tapping the trackpad or touch sensitive display. When the computer detects that the physical switch has been engaged (or that the tapping of the track pad or touch sensitive screen has occurred), it takes the apparent location of the cursor (or in the case of a touch sensitive screen, the detected position of the finger) on the screen and executes the process associated with that location. As an example, not intended to limit the breadth of the disclosed invention, a graphical object that appears to be a two dimensional box with the word “enter” within it may be displayed on the screen. If the computer detects that the switch has been engaged while the cursor location (or finger location for a touch sensitive screen) was within the boundaries of a graphical object, for example, the displayed box, the computer will execute the process associated with the “enter” command. In this way, graphical objects on the screen create a user interface that permits the user to control the processes operating on the computer.

In some instances, especially where the user computer is a mobile computing device used to access data through the network the network may be any type of cellular, IP-based or converged telecommunications network, including but not limited to Global System for Mobile Communications (GSM), Time Division Multiple Access (TDMA), Code Division Multiple Access (CDMA), Orthogonal Frequency Division Multiple Access (OFDM), General Packet Radio Service (GPRS), Enhanced Data GSM Environment (EDGE), Advanced Mobile Phone System (AMPS), Worldwide Interoperability for Microwave Access (WiMAX), Universal Mobile Telecommunications System (UMTS), Evolution-Data Optimized (EVDO), Long Term Evolution (LTE), Ultra Mobile Broadband (UMB), Voice over Internet Protocol (VoIP), Unlicensed Mobile Access (UMA), any form of 802.11.xx or Bluetooth.

Computer program logic implementing all or part of the functionality previously described herein may be embodied in various forms, including, but in no way limited to, a source code form, a computer executable form, and various intermediate forms (e.g., forms generated by an assembler, compiler, linker, or locator.) Source code may include a series of computer program instructions implemented in any of various programming languages (e.g., an object code, an assembly language, or a high-level language such as Javascript, C, C++, JAVA, or HTML or scripting languages that are executed by Internet web-broswers) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The invention may be described in the general context of computer-executable instructions, such as program modules, being executed by a computer. Generally, program modules are comprised of data that is code, which when executed causes the computer to perform various actions, which may be described as source code, scripts, routines, programs, objects, binaries, executable code components that, when executed by the CPU, perform particular tasks or implement particular abstract data types and when running, may generate in computer memory or store on disk, various data structures. A data structure may be represented in the disclosure as a manner of organizing data, but is implemented by storing data values in computer memory in an organized way. Data structures may be comprised of nodes, each of which may be comprised of one or more elements, encoded into computer memory locations into which is stored one or more corresponding data values that are related to an item being represented by the node in the data structure. The collection of nodes may be organized in various ways, including by having one node in the data structure being comprised of a memory location wherein is stored the memory address value or other reference, or pointer, to another node in the same data structure. By means of the pointers, the relationship by and among the nodes in the data structure may be organized in a variety of topologies or forms, including, without limitation, lists, linked lists, trees and more generally, graphs. The relationship between nodes may be denoted in the specification by a line or arrow from a designated item or node to another designated item or node. A data structure may be stored on a mass storage device in the form of data records comprising a database, or as a flat, parsable file. The processes may load the flat file, parse it, and as a result of parsing the file, construct the respective data structure in memory. In other embodiment, the data structure is one or more relational tables stored on the mass storage device and organized as a relational database.

The computer program and data may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed hard disk), an optical memory device (e.g., a CD-ROM or DVD), a PC card (e.g., PCMCIA card, SD Card), or other memory device, for example a USB key. The computer program and data may be fixed in any form in a signal that is transmittable to a computer using any of various communication technologies, including, but in no way limited to, analog technologies, digital technologies, optical technologies, wireless technologies, networking technologies, and internetworking technologies. The computer program and data may be distributed in any form as a removable storage medium with accompanying printed or electronic documentation (e.g., a disk in the form of shrink wrapped software product or a magnetic tape), preloaded with a computer system (e.g., on system ROM or fixed disk), or distributed from a server, website or electronic bulletin board or other communication system (e.g., the Internet or World Wide Web.) It is appreciated that any of the software components of the present invention may, if desired, be implemented in ROM (read-only memory) form. The software components may, generally, be implemented in hardware, if desired, using conventional techniques.

It should be noted that the flow diagrams are used herein to demonstrate various aspects of the invention, and should not be construed to limit the present invention to any particular logic flow or logic implementation. The described logic may be partitioned into different logic blocks (e.g., programs, modules, functions, or subroutines) without changing the overall results or otherwise departing from the true scope of the invention. Oftentimes, logic elements may be added, modified, omitted, performed in a different order, or implemented using different logic constructs (e.g., logic gates, looping primitives, conditional logic, and other logic constructs) without changing the overall results or otherwise departing from the true scope of the invention. Where the disclosure refers to matching or comparisons of numbers, values, or their calculation, these may be implemented by program logic by storing the data values in computer memory and the program logic fetching the stored data values in order to process them in the CPU in accordance with the specified logical process so as to execute the matching, comparison or calculation and storing the result back into computer memory or otherwise branching into another part of the program logic in dependence on such logical process result. The locations of the stored data or values may be organized in the form of a data structure.

The described embodiments of the invention are intended to be exemplary and numerous variations and modifications will be apparent to those skilled in the art. All such variations and modifications are intended to be within the scope of the present invention as defined in the appended claims. Although the present invention has been described and illustrated in detail, it is to be clearly understood that the same is by way of illustration and example only, and is not to be taken by way of limitation. It is appreciated that various features of the invention which are, for clarity, described in the context of separate embodiments may also be provided in combination in a single embodiment. Conversely, various features of the invention which are, for brevity, described in the context of a single embodiment may also be provided separately or in any suitable combination. It is appreciated that the particular embodiment described in the Appendices is intended only to provide an extremely detailed disclosure of the present invention and is not intended to be limiting.

The foregoing description discloses only exemplary embodiments of the invention. Modifications of the above disclosed apparatus and methods which fall within the scope of the invention will be readily apparent to those of ordinary skill in the art. Accordingly, while the present invention has been disclosed in connection with exemplary embodiments thereof, it should be understood that other embodiments may fall within the spirit and scope of the invention as defined by the following claims.

What is claimed:

1. A method executed by a computer system comprised of a machine learning engine for generating an absorbed dose map image data object corresponding to a patient comprising:
Receiving a PHD data set corresponding to the patient;
Receiving an IS data set corresponding to the patient;
Receiving an administered dose value corresponding to the patient;
Inputting the received PHD data set, received IS data set and administered dose value into a machine learning engine trained using known PHD, corresponding known IS data, corresponding known administered dose values as inputs and corresponding known ADMs as an output target variable;
Receiving from the trained machine learning engine output data representing a predicted ADM corresponding to the patient.

2. The method of claim 1 where the administered dose value represents a dosage value of either PSMA INT or PSMA-617.

3. A method executed by a computer system comprised of a machine learning engine for generating an absorbed dose map image data object corresponding to a patient comprising:
Receiving a PHD data set corresponding to the patient;
Receiving a first PIS data set corresponding to the patient, said first PIS data generated after the patient has received a radiopharmaceutical treatment;
Receiving an administered dose value corresponding to the patient;
Inputting the received PHD data set, received first PIS data set and administered dose value into a machine learning engine trained using known PHD, corresponding known PIS data, corresponding known administered dose values as inputs and corresponding known ADMs as an output target variable;
Receiving from the trained machine learning engine output data representing a predicted ADM corresponding to the patient.

4. The method of claim 3 further comprising selecting a target PIS determined at a randomly selected timepoint out of all possible post-injection timepoints in a dosimetry workflow.

5. A method executed by a computer system comprised of a machine learning engine for generating an absorbed dose map image data object comprising:
Generating a PIS using a machine learning engine trained by known PHD and IS as inputs and known PIS as a target variable;
Receiving a PHD data set;
Receiving an IS data set;
Receiving an administered dose value;
Generating a predicted PIS by using a machine learning engine trained using known PHD, corresponding IS data and corresponding administered dose values;
Inputting the generated PIS into a dosimetry workflow process to generate a predicted ADM.

6. A method executed by a computer system comprised of a machine learning engine for generating an absorbed dose map image data object comprising:
Receiving a PHD data set;
Receiving an IS data set;
Receiving an administered dose value;
Generating a predicted PIS using a machine learning engine trained by known PHD and corresponding known IS and administered dose values as inputs and known PIS as a target variable;
Inputting the generated PIS into a dosimetry workflow process to generate a predicted ADM.

7. A method executed by a computer system comprised of a machine learning engine for generating an absorbed dose value data object comprising:
Receiving data selecting an area or volume corresponding to a patient;
Receiving a PHD data set;
Receiving an IS data set;
Generating at least one predicted absorbed dose value corresponding to the selected area or volume using the PHD and IS data as input by using a machine learning engine trained using known PHD, corresponding known IS data and known absorbed dose values.

8. Method of claim 7 where the IS data set is a diagnostic PET scan.

9. The method of claim 1 or claim 5 further comprising:
Generating a predicted PET image by inputting the generated ADM into a machine learning engine trained using PHD, corresponding ADM data and known PET image data as the target variable;
Storing the generated PET image data.

10. The method of claim 9 further comprising:
Inputting the generated PET image data into a process that generates a predicted ADM using PET image data.

11. A method executed by a computer system comprised of a machine learning engine for determining a value of a predicted treatment outcome parameter corresponding to a patient comprising:
Receiving a PHD data set corresponding to the patient;
Receiving an IS data set corresponding to the patient;
Receiving an administered dose value corresponding to the patient;
Inputting the received PHD data set, received IS data set and administered dose value into a machine learning engine trained using known PHD, corresponding known IS data, corresponding known administered dose values as inputs and corresponding known the predicted treatment outcome parameter values as an output target variable;
Receiving from the trained machine learning engine output data representing a predicted treatment outcome parameter value corresponding to the patient.

12. The method of claim 11 where the treatment outcome parameter is one of a numerical time-to-event metric, a probability, a risk score, a survival interval or a progression free survival interval.

13. The method of claim 11 where the treatment outcome parameter is a numerical time-to-event metric value comprised of at least one of prostate-specific antigen and chromogranin levels.

14. The method of claim 11 where the treatment outcome parameter is a probability value of an adverse event.

15. The method of claim 14 where the probability value represents the probability of at least one of: anemia, diarrhea, constipation, fatigue, myelosuppression (i.e., decreased hemoglobin, decreased platelets, decreased leukocytes, and decreased neutrophils), spontaneous and/or uncontrolled episodes of bleeding, infection, sepsis, acute kidney injury and/or severe renal toxicity, electrolyte abnormalities, pain, vertigo, temporary or permanent infertility, embryo-fetal toxicity, leukopenia, nausea, vomiting, hemotoxicity, nephrotoxicity, thrombocytopenia, dry eye, xerophthalmia, dry mouth, xerostomia and secondary malignancy.

16. A method executed by a computer system comprised of a machine learning engine for generating an adverse event prediction value comprising:
Receiving data selecting an area or volume corresponding to a patient;
Receiving data specifying a radiopharmaceutical dosage value;
Receiving a PHD data set;
Receiving an IS data set;
Generate a predicted adverse event value by using a machine learning engine trained using known PHD, corresponding IS data, corresponding known dose rate values and a target output variable corresponding known adverse and non adverse events.

17. A method executed by a computer system comprised of a machine learning engine for generating treatment quality prediction value comprising:
Receiving data selecting an area or volume corresponding to a patient;
Receiving data specifying a radiopharmaceutical dosage value;
Receiving a PHD data set;
Receiving an IS data set;
Generating a predicted treatment quality value by using a machine learning engine trained using known PHD, corresponding known IS data, corresponding known data selecting an area or volume and known treatment outcome values.

18. The method of claim 17 where the treatment quality value is a value representing a tumour volume reduction factor for a specific tumour.

19. The method of claim 17 where the treatment quality value is a value representing a tumour volume reduction factor for the entire body.

20. The method of claim 19 where the treatment quality value is a value representing a magnitude of tumor metabolism.

21. The method of claim 19 where the treatment quality value is a value representing a magnitude of a tumor targeting agent for a specific tumour.

22. The method of claim 19 where the treatment quality value is a value representing a blood level of biochemical markers correlated with tumour activity for a specific tumour.

23. The method of claim 19 where the treatment quality value is a value representing a magnitude of tumor metabolism for the entire body.

24. The method of claim 19 where the treatment quality value is a value representing a magnitude of a tumor targeting agent for the entire body.

25. The method of claim 19 where the treatment quality value is a value representing a blood level of biochemical markers correlated with tumour activity for the entire body.

26. The method of claim 19 where the treatment quality value is a value representing a relative amount of pain perceived by a patient.

27. The method of claim 1 further comprising:
Receiving data representing a region of the patient body; and
Receiving from the trained machine learning engine output data representing a predicted ADM for the region corresponding to the patient.

28. The method of claim 1 where the IS is comprised of a dynamically scanned PET video data.

29. A system comprised of a computer, said computer comprised of a data storage device containing program data that when executed causes the computer system to execute any one of the methods 1-28.
